(19) United States
(12) Patent Application Publication
(10) Pub. No.: US 2010/0092397 A1
(43) Pub. Date: Apr. 15, 2010

(54) Method For Treatment of COPD and Other Pulmonary Diseases

(75) Inventor: Thomas Hofmann, Doylestown, PA (US)

Correspondence Address: PETERS VERNY , L.L.P., 425 SHERMAN AVENUE, SUITE 230, PALO ALTO, CA, 94306, US

(73) Assignee: Activaero GmbH, Gemunden (DE)

(21) Appl. No.: 12/570,305

(22) Filed: Sep. 30, 2009

Related U.S. Application Data
(60) Provisional application No. 61/195,908, filed on Oct. 14, 2008.

(51) Int. Cl. A61K 9/12; A61P 11/00

(52) U.S. Cl. 424/43

(57) ABSTRACT

A method for treatment of patients with pulmonary diseases by providing an aerosolized combination of a methylxanthine and a topical steroid administered into a patient's conducting and central airways. The method utilizes a specific treatment protocol and a nebulizing system providing an aerosol having particles of a predetermined mass medial aerodynamic diameter (MMAD) delivered to the conducting and central lungs with overpressure and under controlled conditions.

This application claims priority of the Provisional application Ser. No.: 61/195,908, filed on Oct. 14, 2008.

### BACKGROUND OF THE INVENTION

1. Field of the Invention

This invention concerns a method for treatment of patients with chronic obstructive pulmonary disease (COPD), severe asthma, steroid dependent asthma, asthma in smokers or in subjects subjected to secondary smoke, cystic fibrosis (CF), idiopathic pulmonary fibrosis (IPF), pulmonary arterial hypertension (PAH) and other similar pulmonary diseases by providing an inhalable aerosol comprising a combination of an aerosolized methylxanthine and a topical steroid. The inhalable aerosol is administered into a patient's conducting and central airways according to a specific treatment protocol comprising administration of an aerosol containing a methylxanthine/steroid combination or a methylxanthine prodrug/steroid combination where the aerosol has particles of a predetermined mass medial aerodynamic diameter (MMAD) sizes between 3 and 8 μm delivered predominantly to the conducting and central lungs with overpressure using a nebulizing system comprising a jet or ultrasonic nebulizer, compressor, an electronic control means and a nebulizing protocol. The nebulizer is combined with airflow control and the aerosol is administered with overpressure. The method results in selective and targeted deposition of a methylxanthine/steroid combination into central and conducting airways. Delivery of the therapeutically effective amount of the drug combination is accomplished in a fast and efficacious manner. The method provides substantial improvement of clinical symptoms in patients suffering from COPD and the other pulmonary diseases with elimination or great reduction of secondary side effect.

2. Background and the Related Disclosures

Pulmonary diseases present a serious problem for many people who are affected. The available treatments for these diseases include administration of steroids. Treatments with steroids are usually problematic because they often lead to undesirable secondary symptoms or to a development of a steroid resistance. So called “steroid resistance” is a well known problem in asthma, COPD, and cystic fibrosis.

All pulmonary diseases that develop steroid resistance are good candidates for treatment according to this invention.

Chronic obstructive pulmonary disease (COPD) is a pulmonary disease that encompasses several conditions. COPD is an all inclusive, non-specific term for chronic symptoms of cough, excessive production of mucus or sputum and dyspnea that may be connected with bronchitis, asthmatic bronchitis or emphysema. COPD thus may cover all of the above or only some of the above conditions but typically this term is used to describe a persistent lung disease with narrowing of the airways and with inflammation. While the bronchitis causes inflammation of the bronchi and/or trachea, emphysema is a further advanced disease resulting in destruction of the alveoli and bronchioles.

A development of COPD is most often attributed to smoking or being subjected to a secondary smoke for a long time. Smoking or secondary smoke damages the lining of the airways leading to inflammation. Inflammation stimulates the damaged lining to secrete abnormal amount of mucus and causes narrowing of airways and airway constriction. Part of the pathophysiology in COPD is also the “steroid resistance”, mediated by a reduced HDAC (histone deacetylase) enzyme functioning.

The underlying conditions of COPD are irreversible and consequently the only treatment that is available for COPD is administration of drugs that alleviate the COPD symptoms and slow down the disease progression.

Among the drugs that are used for treatment of COPD are short or long-acting bronchodilators such as salbutamol or tiotropium or steroid inhalers or steroid tablets. A long-term use of steroids, as is well known, leads to very severe secondary symptoms, such as changes in appearance, acne, weight gain, swelling of face and abdomen, fragile skin, easy bruising, irritability, agitation, euphoria, depression, insomnia, increase in susceptibility to infections, glaucoma, high blood pressure, cataracts, muscle weakness, avascular necrosis of bone and osteoporosis.

It would therefore be advantageous to have available some alternative treatment for COPD that would ameliorate severe secondary symptoms observed with steroid treatment of COPD.

Another pulmonary disease that has often overlapping symptoms with COPD is severe steroid dependent asthma, asthma in smokers and people subjected for a long time to secondary smoke. The only difference between the two diseases is that in COPD the damage to the airways is permanent and irreversible while in asthma the airway narrowing is intermittent and can be reversed with medication, typically comprising steroids, again exposing a patient to undesirable secondary side effects of a steroidal treatment.

Idiopathic pulmonary fibrosis (IPF) is a pulmonary disease that results from an autoimmune disorder or that is an after effect of an infection resulting in the uncontrollable inflammation, immune activity in the lungs and the fibrosis processes. Symptoms of IPF are a dry cough and progressive dyspnea. Eventually, IPF leads to death due to respiratory failure, hypoxemia, right-heart failure, a heart attack, blood clot (embolism) in the lungs, stroke, or lung infection brought on by the disease. The early stages of IPF are marked by alveolitis, an inflammation of the alveoli of the lungs leading to alveoli damage, scarring and fibrosis. The scarring of the alveoli reduces the ability of the lungs to transfer oxygen into the blood causing hypoxemia and further causing increases in the pressure inside the blood vessels of the lungs.

A primary aim of a treatment for IPF is to reduce the inflammation of the alveoli and stop the abnormal process that ends in irreversible fibrosis. Drugs commonly used are prednisone (steroid), various inhaled steroids, and immunosuppressants such as cytoxan (cyclophosphamide).

Another pulmonary disease that can be successfully treated by the instant invention is pulmonary arterial hypertension (PAH), a disorder primarily of small pulmonary arteries which results in a progressive rise in pulmonary vascular resistance and right ventricular failure.

Pulmonary arterial hypertension (PAH) is a type of pulmonary hypertension where the high blood pressure in the blood vessels connecting the heart to the lungs causes changes to the blood vessels that make it difficult for the heart to pump enough blood to the lungs. These changes produce a constant state of high blood pressure in the vessels of the lungs. While the healthy pulmonary artery is open and elastic, allowing blood to flow through easily, in the PAH pulmonary artery, resistance to blood flow increases because the pulmonary artery narrows and stiffens from blood vessel wall thickening, scar tissue, and clotting.

There is no known cure for this disease and the only therapies currently available are those that alleviate the symptoms of this disease, such as, or example, calcium beta blockers, steroids, prostaglandins, anticoagulants and diuretics.

Cystic fibrosis (CF) is another serious pulmonary disease. CF is characterized by an abnormal production and accumulation of airway mucus and the height of the airway surface liquid. As a result of this accumulation, patients develop chronic airway infection and inflammation. The accumulation of the mucus in the lungs results in life-compromising lung infections by Pseudomonas aeruginosa and other pathogens.

The typical cystic fibrosis symptoms are production of thick, viscous mucus secretions in the lungs, repeated infections and inflammations, recurrent pneumonia, chronic cough, bronchitis, asthma, chronic sinusitis and nasal polyps.

A major medical problem in most patients with cystic fibrosis, however, is a loss of lung function. The cystic fibrosis patient experiences a gradual worsening of lung function each year due to recurring infections and inflammations. The recurring lung infections and inflammation typically cause permanent scarring of the cystic fibrosis lungs.

The treatments of CF include administration of antibiotics, bronchodilators, mucolytics and steroids. While these treatments are successful for short periods of time, they are not so successful in treating the disease during cystic fibrosis exacerbations and for long-term therapy because they lead to resistance to antibiotics and to severe secondary symptoms due to continuing administration of steroids.

One common theme in all the above pulmonary diseases is a presence of inflammations in the lungs. The pulmonary inflammation can be treated with anti-inflammatory medications, such as high doses of topical steroids to prevent pulmonary function decline. Side effects of high dose steroids are dose limiting and are well documented with long term administration of topical steroids.

Another common theme in the inflammatory pulmonary diseases is an increased expression of multiple inflammatory genes that are regulated by pro-inflammatory transcription factors, such as, for example NF-kappaB.

Inflammatory gene expression is upregulated by acetylation of core histones through the concerted action of coactivators, such as coactivator CBP (CREB-binding protein), that have intrinsic histone acetyltransferase (HAT) activity and are able to recruit other HAT enzymes. Conversely, gene repression (downregulation) is mediated via histone deacetylases (HDAC) and other corepressors. For example, in biopsies from asthmatic subjects an increase in HAT activity and reduction in HDAC activity was observed. Both upregulation and downregulation of inflammatory gene expression are partially reversible by corticosteroid therapy.

Corticosteroids switch off inflammatory genes in asthma through a combination of a direct inhibition of HAT activity and by the recruitment of HDAC2 to the activated NF-kappaB-stimulated inflammatory gene complex.

In chronic obstructive pulmonary disease (COPD), a corticosteroid insensitive disease, there is a reduction in HDAC activity and HDAC2 expression, which may account for the amplified inflammation resistant to the actions of corticosteroids.

Such reduction in HDAC activity and in HDAC2 expression may be secondary to oxidative and nitrative stress as a result of cigarette smoking and severe inflammation that is observed in asthma, particularly in severe asthma, smoking asthmatic patients and cystic fibrosis patients.

The reduction in HDAC activity induced by oxidative stress can be restored by theophylline, acting through specific kinases, which may be able to reverse steroid resistance in COPD and other inflammatory lung diseases. This action of theophylline was demonstrated with oral administration, however, it is equally or more effective with inhaled application of theophylline and other methylxanthines.

Therefore, the control and upregulation of HAT/HDAC enzymes activity by theophylline/methylxanthine provides a new approach to developing new anti-inflammatory approaches to inflammatory lung diseases.

There are several known mechanisms of action by which methylxanthines act on various enzymes involved in regulation of HAT/HDAC. Methylxanthines act as phosphodiesterase inhibitors. They act as adenosine receptor antagonists. They stimulate release of catecholamine. They inhibit pro-inflammatory transcription factor NF-Kappa B and phosphoinositide 3-kinase. They increase apoptosis. Primarily, however, they increase histone deacetylase activity (HDAC) thereby increasing efficacy of corticosteroids for treatment of pulmonary diseases.

Theophylline, one of the methylxanthines, has been known to reverse the resistance to steroid treatment in pulmonary diseases when administered orally, as disclosed in the following publications. COPD, 2(4):445-55 (2005) describes histone deacetylation as an important mechanism in inflammatory lung diseases. The function of theophylline in chronic obstructive pulmonary disease is described in Proc. Am. Thorac. Soc., 2(4):334-9 (2005) at 340-341. Corticosteroid resistance in airway disease is discussed in Proc. Am. Thorac. Soc., 1(3):264-8 (2004). Theophylline has been shown to restore histone deacetylase activity and steroid responses in COPD macrophages when administered as described in J. Exper. Med., 6: 200(5):689-95 (2004).

While many publications deal with oral or systemic administration of methylxanthines, some attempts were made to deliver the methylxanthines by inhalation.

A number of publications show that it is possible to aerosolize methylxanthines, but at the same time point out to problems connected with such aerosolization. The side effects observed in upper airways upon aerosolization of theophylline and other methylxanthines are described, for example in Aerugi, 44(12):1379-86 (1995). This publication discloses bronchodilating effect of inhalation of aerosolized aminophylline in asthmatic patients. Bronchodilating actions of xanthine derivatives administered by inhalation in asthma are described in Thorax, 40(3):176-9 (1983). This paper describes aerosolization of theophylline at concentration of 10 mg/mL, glycine theophyllinate 50 mg/mL, aminophylline 50 mg/mL, and diprophylline 125 mg/mL. Effect of aminophylline aerosol on the bronchial response to ultrasonic mist of distilled water in asthmatic patients is described in Respiration, 54(4):241-6 (1988). Br. J. Clin. Pharmacol., 14(3):463-4 (1982) describes the use of aminophylline by inhalation at doses of up to 1000 mg of inhaled aminophylline.

All publications cited above tend to show benefit for aerosolized methylxanthines, however, such benefits occurred at very high doses of up to 1000 mg inhaled drug. Thus, while a benefit of bronchodilation and reduction in airway resistance was eventually shown, it was also clearly demonstrated that the bad, intolerable taste and cough produced by the inhaled methylxanthines at these concentrations led to abandonment of this concept, use and further development of these compounds for the inhalation purposes. It has been determined that concentrations of 50 mg/mL of methylxanthine was not tolerated by patients at all, while at 25 mg/ml was somehow tolerated.

As shown from the references above, the dosage needed to obtain some therapeutic benefit was up to 1000 mg of actually inhaled drug. Considering that even at the somehow tolerated concentration of 25 mg/mL, it would be necessary to actually deliver 40 mL of the solution to the lungs at 25 mg/mL and 80 mL of the solution at 50 mg/mL, it is easily understood that such delivery is not practicable or reasonable.

The abstract by Snape et al., ERS (2009) Vienna describes the pre-clinical studies that would seem to support the hypothesis that inhaled low-dose theophylline (ADC4022) administered with an ICS might restore steroid responsiveness in COPD patients. Following 2 week wash-out, subjects (n=47 ADC4022, n=44 placebo) with moderate-severe COPD received 4 week treatment with nebulized budesonide (1 mg twice daily) during the run-in and were then randomized to receive nebulized ADC4022 (12.5 mg, delivered in 10 minutes via Pari Jet nebulizer) or placebo twice daily in addition to budesonide for a further 4 weeks. Obtained results show that lung function was stable in group receiving ADC4022 treatment but declined in the placebo.

Additionally, the U.S. application Ser. No. 11/883,635 filed on Feb. 13, 2006 discloses a combination of methylxanthine compounds and budenoside to treat chronic respiratory diseases by the inhalable route. The combination comprising 250-375 mg of theophylline and 400 μg of budenoside was administered intranasally (in mice) and showed some sparing effect of theophylline when administered together with a steroid.

While these attempts are steps in a right direction, they do not address a number of problems observed with administration of theophylline and methylxanthine generally.

Inhaled theophylline has been shown to have side effects in the upper airways that limit its use for inhalation therapy to very low doses that must be administered in a very short time. When deposited in the upper airways and oropharynx, methylxanthines, such as theophylline, will cause bad, bitter taste that limits its utility in larger amounts. Additionally, it also causes bronchospasm. Moreover, when theophylline is administered via a conventional nebulizer, such as the Pari Jet nebulizer, the lung dose is highly variable, and the beneficial effect of theophylline in the lung cannot be quantified nor consistently delivered. Additionally, when theophylline is administered orally, its plasma levels need to be monitored, as it has side effects such as nausea, tachycardia and other cardiovascular effects and therefore, it is conceivable that such monitoring will be required with large doses administered into the lungs.

It would be, therefore, advantageous to have available and inhaled treatment that would provide effective and quantifiable doses delivered in a short time with high deposition targeted specifically to the airways where the methylxanthine or theophylline would assert its highest effect in accentuating action of a low dose of the steroid.

As discussed above, many pulmonary diseases are typically treated with steroids to overcome inflammation.

Some attempts to treat these diseases with a combination of beta-adrenergic reverse agonists with steroid or xanthine compounds using oral, parenteral or inhalation route are disclosed in U.S. Pat. No. 7,528,175.

Other attempt for treating pulmonary diseases relates to administration of a phosphodiesterase 4 inhibitor in combination with anti-inflammatory corticosteroid by inhalation as described in US patent application 20060035877, published on Feb. 16, 2006.

US application 20070213296, published on Sep. 13, 2007 concerns compositions and methods for treatment of immunoinflammmatory disorders by administering by inhalation Group B adenosine activity upregulator simultaneously with a corticosteroid.

Therefore, it would be advantageous to have available an inhalation method for administration of a selected methylxanthine in combination with a selected steroid substantially into the conducting and central airways of the lungs that would deliver therapeutically effective amount of the drug to provide without concurrent secondary symptoms previously known to be a associated with such delivery.

It is therefore a primary object of this invention to provide a method for efficacious delivery of a methylxanthine/steroid combination into the conducting and central airways using an AKITA® nebulizing system wherein the drug is delivered into the lung with a mild to moderate adjustable pressure in an aerosolized form wherein the aerosol has predominantly particle sizes with a mass median aerodynamic diameter limited to from about 3 to about 8 microns using a controlled slow breathing pattern resulting in high deposition of the drugs in short time between 1 and minutes. The nebulizing system used in this invention enables an effective delivery of methylxanthine/steroid composition substantially into the bronchi and trachea of the conducting and central lungs without substantial deposition of the drug into the oropharyngeal area thereby eliminating the oropharyngeal side effects.

All patents, patent applications and other reference cited herein and hereby incorporated by reference.

### SUMMARY

One aspect of the current invention is a method for treatment of COPD, asthma, cystic fibrosis and other pulmonary diseases by administering to a patient in need thereof a combination of aerosolized methylxanthine selected from the group consisting of theophylline, aminophylline, enprophylline, pentoxyphylline, diprophylline and a phosphodiesterase inhibitor and a topical steroid selected from the group consisting of fluticasone, beclomethasone, budesonide and ciclesonide in an aerosol having a mass medial aerodynamic diameter (MMAD) from about 3 to about 8 microns administered by the jet, ultrasonic, electronic, vibrating mesh or vibrating membrane nebulizer, dry powder inhaler or AKITA® nebulizing system with or without overpressure substantially into the conducting and central lungs wherein said aerosol comprises from about 0.1 mg to about 2 mg of said steroid and from about 25 to about 50 mg of said methylxanthine per day, in combination, dissolved in from about 1 to about 3 ml of solvent of which at least 1 mL comprising at least 0.1 mg of steroid and at 2 to 15 mg of methylxanthine per treatment is deposited in the conducting and central lungs.

Another aspect of the current invention is a method for treatment of pulmonary diseases comprising steps:

preparing a suspension comprising a drug combination of a methylxanthine and a topical steroid, a methylxanthine prodrug and a steroid, or a methylxanthine alone, wherein said suspension comprises from about 0.1 mg to about 2 mg of said steroid and from about 25 to about 50 mg of said methylxanthine dissolved in from about 1 to about 3 mL of a solvent;

aerosolizing said suspension into an aerosol having particle sizes between about 3 and about 8 MMAD;

administering said aerosol to a patient in need thereof using a nebulizing system comprising an electronic or jet nebulizer, a compressor and an electronic control means for controlling an airflow, breathing pattern of a patient and delivery of the aerosol as a bolus according to a treatment protocol, said treatment protocol providing for a slow and controlled breathing pattern of the patient, for controlled airflow of the air or aerosol, for bolus drug delivery and for delivery of said aerosol with efficacy of about 60% to about 70% predominantly into a conducting and central airway with overpressure of 30 mbar or less under controlled conditions; and

delivering said drug combination according to said protocol predominantly into patient conducting and central airways with efficacy of at least 60% deposition of said aerosol in the conducting and central airways,

wherein said treatment results in improvement of pulmonary functions measured by forced expiration flow at 75% of forced vital capacity (FEF75) and in reduction of oropharyngeal deposition and reduction in methylxanthine or steroid side effects.

Another aspect of the current invention is a method for delivery by aerosolization of a combination of methylxanthine with a steroid wherein said delivery of said combination results in diminishing of side effects of methylxanthine and in enhancing of a steroid effect and wherein said delivery results in improvement of pulmonary functions in patients with COPD and other pulmonary diseases.

Still another aspect of the current invention is a method for treatment of COPD, asthma, cystic fibrosis and other pulmonary diseases by administering to a patient in need thereof a combination of aerosolized theophylline and a topical steroid selected from the group consisting of prednisone, fluticasone, beclomethasone, budesonide and ciclesonide, in an aerosol having a mass medial aerodynamic diameter (MMAD) from about 3 to about 8 microns administered by the jet, ultrasonic, electronic, vibrating mesh or vibrating membrane nebulizer, dry powder inhaler or AKITA® nebulizing system with or without overpressure substantially into the conducting and central lungs wherein said aerosol comprises from about 0.1 to about 2 mg of said steroid and from about 3 to about 50 mg of said theophylline, in combination, dissolved in from about 1 to about 4 ml of solvent of which at least 0.5 mL comprising at least 50 ug of steroid and at least 5 mg of theophylline is deposited in the conducting and central lungs. Yet another aspect of the current invention is a method for delivery by aerosolization of a combination of theophylline with a steroid wherein said delivery of said combination results in diminishing of side effects of theophylline and in enhancing of a steroid effect and wherein said delivery results in improvement of pulmonary functions in patients with COPD and other pulmonary diseases.

Still yet another aspect of the current invention is a method for treatment of COPD, asthma, cystic fibrosis and other pulmonary diseases by administering to a patient in need thereof a combination of an aerosolized methylxanthine phenylphosphate prodrug and a topical steroid selected from the group consisting of prednisone, fluticasone, beclomethasone, budesonide and ciclesonide in an aerosol having a mass medial aerodynamic diameter (MMAD) from about 3 to about 8 microns administered by an AKITA® nebulizing system with overpressure by low inhaled airflow substantially into the conducting and central lungs wherein said aerosol comprises from about 0.1 mg to about 2 mg of said steroid and from about 5 to about 50 mg of said methylxanthine prodrug, in combination, dissolved in from about 1 to about 4 ml of solvent of which at least 0.5 mL comprising at least 50 ug of steroid and at least 5 mg of methylxanthine is deposited in the conducting and central lungs.

Another aspect of the current invention is a method for delivery by aerosolization of a combination of methylxanthine phenylphosphate prodrug with a steroid wherein said delivery of said combination results in diminishing of oral and topical side effects of methylxanthine.

Still another aspect of the current invention is a method for treatment of COPD, asthma, cystic fibrosis and other pulmonary diseases by administering to a patient in need thereof a combination of aerosolized methylxanthine selected from the group consisting of theophylline, aminophylline, enprophylline, pentoxyphylline, diprophylline and a phosphodiesterase inhibitor in an aerosol having a mass medial aerodynamic diameter (MMAD) from about 3 to about 8 microns administered by an AKITA® nebulizing system with overpressure by low inhaled airflow substantially into the conducting and central lungs wherein said aerosol comprises from about 5 to about 50 mg of said methylxanthine, dissolved in from about 1 to about 4 ml of solvent of which at least 10 mg of methylxanthine is deposited in the conducting and central lungs.

Still yet another aspect of the current invention is a method for delivery by aerosolization of a combination of methylxanthine with a beta-agonist, anticholinergic, cromone or leucotriene inhibitor.

### DEFINITIONS

As used herein:

“MMAD” means mass median aerodynamic diameter.

“Methylxanthine drug” or “methylxanthine” means a methylxanthine selected from the group the group consisting of theophylline, aminophylline, enprophylline, pentoxyphylline, diprophylline and a phosphodiesterase inhibitor.

“Steroid drug” or “steroid” means a topical steroid selected from the group consisting of prednisone, fluticasone, beclomethasone, budesonide, mometasone and ciclesonide

“Conducting lungs” and “central lungs”, means bronchi and trachea of the lungs fibrosis. Selective deposition of an inhalable methylxanthine and steroid combination in this area contributes to improvement of symptoms of COPD and other pulmonary diseases.

“One breath” means a period of time when a person inhales (inspires) and exhales during a regular breathing pattern that includes inhaling and exhaling.

“Inspiration time” or “inspiration phase” means a fraction of one breath when a person inhales an air or, in this instance, an aerosolized methylxanthine/steroid combination. For purposes of this invention, the aerosolized methylxanthine/steroid combination is administered to a patient in need thereof during the second period of the inspiration time either with a mild or moderate overpressure up to 30 mbar to force the aerosol to the lower lungs using the AKITA® protocol and nebulizer, or as a second volume between the first and second volume of delivered air without particles using a breath actuated nebulizer and protocol.

“Expiration time” means a fraction of one breath when a person exhales the air, nitric oxide or another metabolite from the lungs. For the purposes of this invention, it is preferable that the aerosolized methylxanthine/steroid combination is forced with a mild or moderate overpressure into the central and conducting lungs during inspiration and that it is not exhaled during expiration time or that only a small portion is exhaled.

“Bolus technique” means transportation of the aerosol containing a methylxanthine/steroid combination to a predefined region in the lungs.

“Particle-free air” means the air that does not contain any drug and is delivered before and after the aerosolized drug delivery.

“Overpressure inhalation” means inhalation with actively provided air that is preferably predefined in an airflow for a predefined time. During inspiration the patient adjusts to the inspiratory flow rate. If the patient inhales more passively an overpressure of up to maximum 30 mbar is applied during the inhalation phase to reduce the inspiratory effort. Consequently, the patient is able to inspire a larger deep inhalation volume and inhale with a slower inspiration flow rate compared to a spontaneous inhalation.

“FEF” means forced expiratory flow.

“FVC” means forced vital capacity.

“VC” means vital capacity.

“FEV” means forced expiratory volume.

“FEV1” means forced expiratory volume in one second.

“PFT” means pulmonary function testing measures the function of lung capacity and lung and chest wall mechanics to determine whether or not the patient has a lung problem. Pulmonary Function Tests are commonly referred to as PFTs. When a patient is referred for PFT's, it means that a battery of tests may be carried-out including simple screening spirometry, static lung volume measurement, diffusing capacity for carbon monoxide, airways resistance, respiratory muscle strength and arterial blood gases.

“MEF” means maximal expiratory flow.

“Predominantly” means at least 70-90%.

“Substantially” means at least 44%.

### DETAILED DESCRIPTION OF THE INVENTION

The current invention concerns a method for treatment of various pulmonary diseases such as chronic obstructive pulmonary disease (COPD), severe, steroid resistant asthma, asthma in smokers, cystic fibrosis, idiopathic pulmonary fibrosis, pulmonary arterial hypertension and other similar pulmonary diseases typically treated for extensive periods of time with large doses of steroids. The method provides a means for overcoming problems connected with such long-term treatments with steroids by providing an inhalable aerosol comprising a combination of an aerosolized methylxanthine and a topical steroid.

The methylxanthine/steroid combination is administered into a patient's conducting and central airways as an inhalable aerosol according to a specific treatment protocol providing for efficacious delivery of the methylxanthine/steroid to a specific region of the lungs, namely predominantly to the central and conducting airways. The treatment protocol provides for a preparation of the aerosol having particle sizes of predetermined mass medial aerodynamic diameter (MMAD) between 3 and 8 μm delivered predominantly to the conducting and central lungs with or without overpressure using a jet, ultrasonic, electronic, vibrating porous plate, vibrating mesh nebulizer or energized dry powder inhaler. The jet or electronic nebulizers may further be combined with airflow control and the aerosol may be administered with overpressure. The method results in substantial improvement of clinical symptoms in patients suffering from COPD and the other pulmonary diseases.

The method utilizes nebulization devices and systems allowing individualization of a delivered volumetric flow and vaporized aerosol together with a controlled airflow and with airflow overpressure conditions into a treatment protocol suitable for treatment of inflammatory pulmonary diseases. Such individualized treatment protocol provides for a shift in deposition pattern of the nebulized drug substantially to the conductive and central lungs according to the so called AKITA® treatment protocol.

The AKITA® treatment protocol comprises treating the inflammatory pulmonary disease with administration of a combination of methylxanthine/steroid in an aerosol having particles of a mass median aerodynamic diameter (MMAD) limited to sizes between 3 and 8 μm, that is in particle sizes that are predominantly deposited in the lung central and conductive region. The AKITA® protocol also utilizes a breathing pattern achieved with a slow and controlled inhalation provided by the nebulizing system, also called AKITA® nebulizing system.

Overall, the method of the invention is able to deliver effective dose of the methylxanthine/steroid combination, theophylline/steroid combination, or methylxanthine prodrug/steroid combination during one to three minutes inhalation with effective control of methylxanthine or theophylline side effects. In case of theophylline bad taste side effects that limit its utility, only 25 mg/mL theophylline formulation is needed for high deposition of the drug using the breath control and slow inhalation to overcome bad taste of theophylline.

The nebulizing system comprises components, such as a jet or electronic nebulizer, a compressor and an electronic control means that cumulatively have properties that enable control of the breathing pattern by asserting a positive pressure (also called NIPPV) during the inhalation. This pressure reduces the need for active breathing in COPD patients, which results in much more effective and easier lung delivery of the drugs combination to COPD patients having difficulty breathing or who are unable to breathe without oxygen.

The system also provides for easy determination of actually delivered amount of the drug combination and therefore quantification of such delivery because only a minimal volume containing a smallest possible concentration of the drugs is needed and actually administered only to a site of action.

All other devices known and used for these purposes require higher amounts, higher volumes and more active breathing effort on the patient side and still not accomplish such accurate and effective deposition. Using such a non-accurate delivery device, the benefit of providing methylxanthine or theophylline in conjunction with steroids is lost, inaccurate and does not result in elimination of side effects such as bad taste and bronchospasm.

The current nebulizing system is further made more practicable by providing a small, handheld device storing either the methylxanthine/steroid combination or methylxanthine prodrug/steroid combination and using a miniaturized breath controlling and airflow control means alone or together with, for example vibrating mesh nebulizers to maximize lung deposition.

The method also introduces a methylxanthine and theophylline prodrugs that are delivered in the manner described above. The prodrug, in combination with a steroid is delivered by inhalation into conducting and central airways where it is enzymatically converted into the methylxanthine, and/or specifically, to thephylline.

I. Pulmonary Diseases and Steroid Resistance

The method according to the current invention is intended for treatment of pulmonary diseases that became steroid resistant.

A. Pulmonary Diseases

Pulmonary diseases that are primarily treated with steroids for long periods of time are chronic inflammatory pulmonary diseases where the inflammation is either the cause of the disease or one of the symptoms of the disease. Because of the length of time of treatment many of these diseases become steroid resistant. Inflammatory pulmonary diseases that are candidates for treatment according to this invention are chronic obstructive pulmonary disease (COPD), severe asthma, asthma in smoking patients or in asthmatic patients subjected to the secondary smoke, cystic fibrosis, idiopathic pulmonary fibrosis, pulmonary arterial hypertension where the patients suffering from any of such disease have developed steroid resistance.

The method of the invention provides a means for overcoming this steroid resistance by the effective co-administration of a methylxanthine, preferably theophylline, to the central and conducting airways, along with the steroid by inhalation to enable an effective treatment of pulmonary disease.

B. Therapeutic Methylxanthine/Steroid Combination

A therapeutic combination according to this invention comprises two different types of drugs co-administered in an inhalable aerosol. Both types of drugs have been previously identified as anti-inflammatory agents. However, given individually in therapeutically effective dosages, both types assert severe secondary side effects.

The first type of drug is a topical steroid selected from the group consisting of prednisone, fluticasone, beclomethasone, budesonide, mometasone and ciclesonide. A suitable topical steroid for treatment of each of the pulmonary disease depends on the disease to be treated, on the patient's tolerance or degree of resistance and on the stage and severity of the disease. The aim of this invention is to use a most effective steroid for the treatment of the disease in the smallest possible dose sufficient to achieve a desirable therapeutic effect without pronounced side effects. The dosage used in the aerosolable combination differs depending on the circumstance but generally, the combination will contain from about 0.1 to about 2 mg of the steroid, with preferred amount of the steroid deposited in the lungs being between 0.1 mg to about 1.5 mg.

The second type of drug is a methylxanthine selected from the group the group consisting of theophylline, aminophylline, enprophylline, pentoxyphylline, diprophylline and a phosphodiesterase inhibitor. The most preferred methylxanthine is theophylline. The dosage of methylxanthine in the aerosolizable combination is from about 2 to about 50 mg, with preferred amount deposited into the lungs being between about 2 and about 5 mg. This dose is small enough to overcome problems of the local intolerance of inhaled methylxanthine resulting in cough, bad taste and bronchospasm observed with administration of larger amounts of these compounds (Am. J. Respir. Crit. Care Med., 167: 813-818 (2003).

Theophylline is an effective pulmonary drug with a narrow therapeutic window that requires strict monitoring of plasma levels. The recommended effective range is 10-20 mg/L plasma level. Levels above this range reached upon systemic (oral or i.v.) administration result in headache, nausea, vomiting, abdominal discomfort, restlessness, increased acid secretion, gastroesophageal reflux, and diuresis. At higher concentrations, convulsions, cardiac arrhythmias, and death may occur. Additionally, theophylline and other methylxanthines also interfere with the CYP 450 hepatic metabolism of multiple drugs. The use of methylxanthines is therefore strictly limited to a safe range of under 20 mg/L plasma levels. The plasma levels achieved under the current invention are between 1 and 3 mg/L, or even less.

An important additional aspect of the invention is the use of methylxanthine prodrugs, such as, for example, a substituted phenylphosphate. When delivered to the lung, endogenous enzymes present in the lung tissue and airway degrade such prodrug into a corresponding methylxanthine. Depending on the prodrug, the methylxanthine prodrug is converted in the lungs into theophylline, aminophylline, enphylline or pentoxyphylline. In this embodiment, the methylxanthine prodrug, rather than methylxanthine, is combined with a steroid and delivered into lungs according to the method of the invention.

This approach provides a means for overcoming problems and disadvantages connected with the adverse side effect profile of ICS (inhaled corticosteroids), namely candidiasis, sore throat and dysphonia, and of methylxanthines, namely cough, bad taste and tachycardia, by providing a water-soluble, steroid/methylxanthine prodrug to mask the pharmacological properties of steroids and particularly methylxanthines until such a prodrug reaches lungs, thereby mitigating the oropharyngeal side effects of ICS and multiple side-effects of methylxanthines.

In the lung, the prodrug is metabolized by alkaline phosphatase into the active form of methylxanthine. The alkaline phosphatase is absent in the mouth and pharynx and, therefore, the bad taste and side effects of methylxanthine are absent in the mouth and pharynx, with methylxanthine available to the lungs after conversion of the prodrug.

The methylxanthine prodrug incorporates charged phosphate and quaternary ammonium groups, which renders the molecule highly polar and water soluble and imparts its affinity to lung DNA and protein thus minimizing rapid systemic absorption, as well as absorption due to swallowing. Furthermore, since the prodrug cannot be activated in absence of alkaline phosphatase and since this enzyme is not present in oropharyngeal area, the oropharyngeal and systemic side effects are eliminated.

The prodrug/steroid composition is formulated as either a liquid or dry powder. The formulation is suitable for delivery of the prodrugs to the lung airways in an aerosol having a mass median average diameter predominantly between 3 and 8μ. The formulated and delivered efficacious amount of a substituted phenylphosphate prodrug is sufficient to deliver therapeutic amounts of both methylxanthine and steroid for the treatment of pulmonary diseases.

The invention therefore uses novel approaches to overcome the previously observed problems with both the steroid resistance and with methylxanthine treatment side effects.

First, the topical intolerance to inhaled methylxanthines is overcome by an airflow control and particle size design of the aerosol device and its ability to deliver the combination into the lung conductive and central region where it is most effective without loss of much drug in the oropharyngeal area.

Second, the consistent drug deposition into the lung will reduce the size and cost of the necessary clinical studies. Third, the therapeutic effectivity of both the steroids and methylxanthines is increased by their combination of the methylxanthine with the steroid. In this combination the concentration of each drug is much smaller than when administered individually with both drugs released in relevant site of action in the lungs.

Third, the methylxanthine prodrug/steroid combination further improves chances for achieving higher concentrations of the methylxanthine in the lungs by delivering this combination to the relevant site in the lungs where the resident lung enzymes cleave the methylxanthine prodrug, such as for example, a substituted phenylphosphate, into the active drug.

Since the steroid enabling effect in the lungs is present at low systemic levels of 10⁻⁶ to 10⁻⁵M, and the enabling effect of methylxanthine is present at systemic levels below 10 mg/L, these levels can be topically reached via aerosolization of methylxanthine/steroid combination according to the invention.

II. Method for Treatment of Pulmonary Diseases

A method for treatment of the pulmonary disease comprises administration of a combination of a steroid with a methylxanthine, preferably theophylline, to patients as a nebulized aerosol having particle sizes of controlled homogeneous sizes corresponding to sizes of trachea and bronchi in the conducting and central airways, using an electronic nebulizer (AKITA® nebulizing system) modified with means to allow a slow and controlled breathing pattern with aerosol bolus delivered at a beginning of nebulization.

This system permits delivery of the aerosol predominantly into the conducting and central airways of the lungs according to a specifically designed and individualized protocol that controls breathing pattern of a treated patient.

B. Aerosol

The aerosol used for treatment of the pulmonary diseases comprises a depositing a combination of a topical steroid and methylxanthine or methylxanthine prodrug preferentially into the conducting and central airways. The combination is aerosolized into particle sizes limited to between about 3 and about 8 μm, with a predominant number of at least 70% but preferably 90% of these particles being within this range.

Before aerosolization, the combination alpha is dissolved in saline or sterile water in concentration as described above. Typically, the nominal dose is placed in 1 to 5 ml of the solvent. The solution of the combination is aerosolized and delivered as an aerosol into the conducting and central airways.

C. Lung Deposition

The resulting aerosols are deposited in both the central and conducting airways using the AKITA® nebulizing system due to impaction. Impaction is the main deposition mechanism in the central airways. The particles above 3 μm have higher velocity, and are, therefore, more likely to impact.

The deposition mechanisms for the drug delivery into the peripheral airways of the lower lungs depends on the number of particles present in the aerosol and on their sizes as well as on their distribution and deposition into the central and conducting airways of the lungs as well as on breathing pattern of the patient.

However, the size of the particles and the normal breathing pattern alone is not sufficient to deliver sufficient amount of the drug to the conducting and central lungs of the patient unless the particle deposition is somehow enhanced. Without such enhancement, the particles will be deposited only according to their sizes in the area of the lung that has corresponding sizes as well as in other areas, particularly in oropharyngeal region. Such deposition, however, will not happen in patients with pulmonary diseases as their lungs are impaired due to the disease and, unless there are some conditions that would permit overcoming such impairment.

The current method and devices disclosed herein provide such conditions by delivering the combination under mild or moderate overpressure and by regulating a breathing pattern during such delivery according to the AKITA® nebulizing protocol.

D. Therapeutic Nebulizing Protocol

Therapeutic nebulizing protocol, also called AKITA® nebulizing protocol, for treatment of pulmonary diseases comprises a preparation of aerosol of appropriate sizes to increase the efficacy of the deposition of the steroid/methylxanthine combination in the central and conducting airways of pulmonary disease patients, delivery of said aerosol into said central and conducting airways using a jet or electronic nebulizer, a slow inhalation of the aerosol with aerosol bolus at start of each breath and a clinical evaluation of the patient following the inhalation treatment.

Preparation of the Aerosol

The aerosol having the optimal particle sizes for homogenous deposition of the drug in the peripheral airways of the lower lungs that prevents high losses of drug in the oropharynx is prepared from a solution of the combination of the steroid and methylxanthine by nebulizing from about to about 5 mL of said solution into an aerosol of appropriate sizes between about 3 to 8, preferably at least 90% of the aerosol particles having these sizes, to increase the efficacy of the steroid/methylxanthine deposition targeted to the conducting and central airways of patients.

2. Delivery of the Aerosol

Delivery of the steroid/methylxanthine into the central and conducting lungs using the jet or electronic nebulizer equipped, optionally, with a vibrating mesh or vibrating membrane according to the therapeutic nebulizing protocol is achieved in less the 10 minutes, preferably in from about 1 to about 5 minutes and most preferably in about 2 minutes. Treatment is administered several times a day, as needed, but is preferably limited to once or twice.

3. Slow Inhalation with Aerosol Bolus

The patient's breathing pattern during the delivery of steroid/methylxanthine to the central and conducting airways is as important as is the size of the aerosol particles of the nebulized steroid/methylxanthine.

The breathing pattern used to inhale the aerosol influences the deposition of particles in the respiratory tract. High inspiratory flow enhances the impaction of particles, and thus enhances a more central deposition. Low inspiratory flow enables particles to penetrate more deeply into the lung. Such controlled breathing pattern is enabled by using AKITA® nebulizing system.

An important aspect of the invention, therefore, is the ability of the AKITA® therapeutic nebulizing system to provide controlled conditions for patient's breathing pattern permitting a slow inhalation and, at the same time, providing aerosol bolus delivering larger dosages of the drug at start of each breath in a slow and protracted breathing inhalation maneuver.

The slow inhalation maneuver preprogrammed by therapeutic nebulizing system, using the jet or electronic nebulizer and delivering an aerosol comprising steroid/methylxanthine aerosolized into particle sizes predominantly of about 3 to 8 μm MMAD, enables aerosolized particles to penetrate deeply into the periphery of the lower lungs and provides a better peripheral lung deposition in patients with cystic fibrosis.

Such slow breathing pattern is limited to breathing volume of from about 50 to about 300 mL/second with inhalation volume limited to about 300 to about 1500 mL, applied with a mild to moderate overpressure up to 30 mbar.

Typically the inhalation involves a delivery of from 1 to about 5 mL, preferably from about 1 to about 2 mL, of steroid/methylxanthine with deposition of the steroid/methylxanthine of at least 1 mg for methylxanthine and 75 μg for the steroid, preferably all this amount being deposited into the central and conducting airways.

The slow inhalation method defines a partition of one breath into two fractions, namely an inspiration time and expiration time wherein during the inspiration time a so called bolus technique is used to transport the drug containing aerosol to a predefined region in the lungs and, during the expiration time, to exhale a minimum of the drug from the lungs at end of the breath.

The method of the invention results in four to five times higher deposition and delivery of steroid/methylxanthine combination, using the therapeutic nebulizing system compared to deposition achieved with other currently used nebulizers, into the central and conducting lungs of the patient in less than 2 to 4 minutes, in average, during the slow inhalation.

4. Clinical Evaluation

Clinical evaluation of the patient following the inhalation treatment with steroid/methylxanthine according to the method of the invention includes but is not limited to spirometry, oxygen saturation parameters and profile, determination of forced expiratory flow (FEF75), forced expiratory volume (FEV1), forced vital capacity (FVC), pulmonary function testing (PFT) and maximal expiratory flow (MEF25 or MEF75).

5. Deposited Doses

The current method enables deposition of about four to five times more of the filling dose of the drug placed in the nebulizer in the central and conducting airways of the lungs compared to the other conventional nebulizers in shorter time and with eliminated or much lesser secondary side effects.

E. Therapeutic Nebulizing System

Therapeutic nebulizing system (AKITA® nebulizing system) provides means for controlling both the aerosolization of the drug into the particles having predominantly sizes in the range from about 3 to about 8 μm with majority of at least 90% of particles having size from about 3 to 8 μm MMAD.

Using this system, these particles are deposited in the central and conducting airways of the lungs, bronchi or trachea that have sizes in this range. However, even provided that the aerosol having MMAD of these sizes may be prepared, it is still very difficult to deliver such aerosol into pulmonary diseases, when the lungs are impaired, constricted and often filled with mucus and inflamed. All these factors provide a natural barrier and resistance to the deposition of the drug there. Consequently, some intervention means that would permit overcoming this problem is necessary.

The AKITA® nebulizing system is equipped with a means to deliver the aerosol into such impaired lungs under mild or moderate overpressure of about and up to 30 bars. With this overpressure, the aerosolized drug is gently pushed into the patient's lungs and deposited primarily in the central and conducting airways of the lower lungs. The AKITA® nebulizing system additionally provides a means for influencing a breathing pattern of the patient, which is another contributing factor to the improvement of delivery of the steroid/methylxanthine combination into the lungs of patients.

The therapeutic nebulizing system used for treatment of pulmonary diseases is therefore able to deal with all important factors that can influence treatment of these diseases. The system influences the steroid/methylxanthine combination administration by providing the aerosol targeted primarily to the central and conducting lungs delivered under mild or moderate overpressure under conditions that control a breathing pattern of the pulmonary disease patient.

The therapeutic nebulizing system comprises an electronic or jet nebulizer, a compressor and an electronic control means for controlling an airflow, breathing pattern of a patient and delivery of the aerosol as a bolus according to a treatment protocol, said treatment protocol providing for a slow and controlled breathing pattern of the patient, for controlled airflow of the air or aerosol, for bolus drug delivery and for delivery of said aerosol with efficacy of about 60% to about 70% predominantly into a conducting and central airway with overpressure of 30 mbar or less under controlled conditions.

The therapeutic nebulizing system is approved for all available liquid inhalation medications. The use of a personalized smart card ensures that treatment with the therapeutic nebulizing system is adjusted to the individual requirements of each patient. For a central and conducting airways deposition, a relatively small particle size of about 3 to about 8 μm and a slow, deep inhalation maneuver is used.

The therapeutic nebulizing system comprises of a compressor unit, the nebulizer and the electronic control means set together thereby providing a highly effective inhalation system for inhalation therapy of the pulmonary diseases.

The AKITA® system comprises preferably the AKITA®2 compressor and AKITA®1 jet or, preferably, the AKITA®2 electronic nebulizer. The AKITA®2 nebulizer is able to generate particles with a MMAD from 3.0 μm to 8.0 μm and a GSD of 1.6.

AKITA®2 nebulizer operates under following parameters:

Noise emission: <70 dB(A)

Operating voltage: 230V±10%, 50 Hz, 0.7 A

Suction trigger pressure: −1.0 to −4.0 mbar

Inhalation flow: 50-300 ml/sec, adjustable, using SmartCard

Flow pattern: Constant inspiration flow

Nebulizer pressure: 3 mbar, adjustable, using SmartCard

Ambient conditions: 5 to 40° C.

10 to 95% relative humidity

600 to 1100 hPa atmospheric pressure

Particle Sizes of the Aerosol

The therapeutic nebulizing system provides an aerosol having the optimal particle sizes for homogenous deposition in the central and conducting airways of the lower lungs that prevents high losses of the drug combination in the oropharynx as well as losses in the lower lungs.

The system provides an aerosol having sizes of aerosolized particles corresponding substantially to a size of the trachea and bronchi. The right particle size for targeting the trachea and bronchi is between 3 and 8 microns. Particles larger than 3 μm are selectively deposited in the more central and upper lungs, namely bronchi and trachea, but they can also be deposited in the mouth and throat, i.e. oropharyngeal area, if there are no conditions controlling such deposition. The nebulizing system provides conditions that limit deposition of the drug in the oropharyngeal area by controlling the breathing pattern and by delivering the drug in bolus aerosol.

Consequently, the method provides for aerosol to be limited to particle sizes between 3 and 8 μm, MMAD, with geometric standard deviation (GSD) of less than 2.5, preferably GDS of about 1.6.

Furthermore, constriction of the bronchi or trachea, edema of the airway walls, mucus, sputum or lower pulmonary bronchoconstriction cause narrowing of the airway diameter and, consequently, the inhaled aerosol would be largely deposited in oropharyngeal area rather then in central and conducting airways. This is often observed in pulmonary disease patients patents during the severe conditions or during the exacerbation of the disease, when there is often increased airway obstruction due to constrictions, infections and inflammations. Consequently, it is important to assure that the treatment of the pulmonary disease with inhalation treatment is targeted strictly to areas where the problems occur and treatment is needed.

3. Delivery of the Aerosol under Overpressure

As discussed already above, delivery of the steroid/methylxanthine combination by an aerosol without any enhancement results in wasting the drug and low efficiency of such delivery as well as deposition of the drug into the oropharyngeal area. Such enhancement is provided for by the instant method by delivering the drug containing aerosol under mild or moderate overpressure. This mild overpressure is particularly important in patients with COPD, and other lung diseases (such as severe asthma, CF) with breathing impairments.

The system provides means to deliver the steroid/methylxanthine combination containing aerosol under overpressure no higher than 30 mbar. Such mild to moderate overpressure allows the aerosol to be actively forced to the central and conducting airways of the lungs even when impaired without causing damage to the lungs.

Such overpressure is achieved with an AKITA® compressor with or without pump unit attached to the AKITA® nebulizer where such unit is optionally further equipped with a timer so that the overpressure period is limited strictly to a fraction of the inspiration time when the steroid/methylxanthine combination aerosol is delivered and, moreover, therapeutic nebulizing system has a safely means shutting off the pressure at 30 mbar.

In one embodiment, the overpressure is initiated by a patient's inspiration time breathing. When the patient inspires with overpressure, the patient's breathing effort is reduced and the patient is able to breath in a deeper and slower breathing pattern. That makes a great difference when compared to a spontaneous inhalation administered without overpressure. The overpressure is preset and regulated according to the treatment protocol.

During the inhalation, the therapeutic nebulizing system provides an overpressure of up to 30 mbar under which the aerosol is administered. Such overpressure allows preferable deposition of the aerosolized drug in the peripheral airways of the lower lungs and also prevents the aerosol removal during expiration because during expiration, the overpressure is not applied and the patient thus exhales normally, without any airflow or pressure being applied.

4. Bolus Technique

The therapeutic nebulizing system and a method for use thereof defines a partition of one breath into two fractions, namely an inspiration time and expiration time wherein during the inspiration time a bolus technique is used to transport the drug containing aerosol to a predefined region, in this case to the central and conducting airways of the lungs, and during the expiration time, to expire a minimum of the drug from the lungs.

In some embodiments of the bolus technique, the inspiration time may be further divided into subfractions where the particle free air is delivered before and after the aerosol delivery of the steroid/methylxanthine combination.

5. Delivery Time

The system provides for shorter delivery time than conventional nebulizer for the same drug amount deposited in the lungs. Typically, the inhalation delivery of the steroid or methylxanthine individually as an aerosol would take at least 20 minutes using a conventional nebulizer and results in deposition of only about one fifth to one tenth of the volume. The current method provides for a deposition of both drugs, in combination, in an aerosol into the lungs in less than 10 minutes, preferably in less than four minutes, resulting in delivery of more than four to five times more of steroid/methylxanthine combination per one treatment.

Although it would be possible perhaps to deliver, by inhalation, the steroid/methylxanthine combination in an aerosol having limited sizes of particles, using other conventional nebulizers, the method, devices and protocol according to the invention provided herein result in a very substantial improvement of delivery and deposition of the steroid/methylxanthine drug combination in the central and conducting lung of the patients. The efficacy of the drug combination deposition is four to five time higher then the one obtained with conventional nebulizers.

III. Treatment Protocol for Treatment of Pulmonary Disease

The actual treatment protocol (AKITA® protocol) for treatment of pulmonary diseases according to the invention consists of several steps that need to be undertaken.

Inhalation System for Control of Breathing Pattern

When the AKITA® protocol is selected for treatment, the patient is provided with the therapeutic nebulizing system, as described below.

The steroid/methylxanthine combination in the predetermined volume of about 1 to about 5 mL containing the steroid/methylxanthine combination in predetermined ranges filled into the nebulizer. For example, 2 mL of steroid/methylxanthine combination is filled in the nebulizer in form an aqueous suspension.

The nebulizer is directly connected with the mouthpiece that is further equipped with pressure sensor connected with a compressor. Inhalation period (inspiration time) is preset to a pattern comfortable for a patient, for example, from 1 to about 10, preferably about 3-4 seconds, of inspiration time. When the inspiration time is not preset, patient's own breathing rhythm controls the inspiration time.

When the patient inhales from the mouthpiece, the pressure sensor responds and starts inhalation by providing a positive overpressure or opening of an inspiration valve. The nebulizer, or an aerosol system, is supplied with compressed air overpressure of up to maximum 30 mbar and the steroid/methylxanthine combination is aerosolized and discharged as an aerosol at a preselected flow rate of about 50-300 mL/sec and with a preselected overpressure. The overpressure lasts for the entire inspiration time. When the inspiration time is preselected for a certain period of time, the overpressure is automatically stopped or shut off at the end of this period because the compressed air supply is interrupted at the end of the inspiration time.

After a period allocated for exhaling, the process is repeated on and off for the entire period of inhalation, preferably for less than 6 minutes. During the inhalation time, the whole dose is preferably aerosolized with only some small residue remaining in the nebulizer.

Electronic equipment that may be attached to the nebulizer permits recordation of the inhalation process, storing all records regarding the dose, time, air flow and overpressure for further optimization of the treatment.

When this method of delivery is selected, during the inspiration time the aerosolized steroid/methylxanthine combination is forced under the overpressure into the peripheral airways of the lower lungs. When the overpressure is withdrawn and the patient exhales, the drug forced into the central lungs is not easily displaced and remains there resulting in substantially higher deposition of the steroid/methylxanthine combination and therefore stronger anti-inflammatory action in the central and conducting airways of the lungs than would happen with a normal breathing pattern without overpressure.

During the exhalation time, the small amount of the steroid/methylxanthine combination that is exhaled is the one that was in the upper lungs at the last moment of the inspiration time. Some fraction of this small amount may be deposited in the oropharyngeal area but most of the drug is exhaled to the outside of the mouth.

B. Breath Actuated Treatment Protocol

The second method for treatment of COPD (and other pulmonary diseases) comprises use of a nebulizing system that is actuated by patient's breathing and comprises the use of a breath actuated nebulizer.

This nebulizer permits depositing aerosolized particles to specific areas of the lung by regulating aerosolization parameters of the device and by instituting a three prong inspiration time delivery.

Using breath actuated nebulizer system, the steroid/methylxanthine combination, in the predetermined amount and volume, as already described above, is filled into the drug cartridge connected with the nebulizer that includes the mouthpiece and a spirometer.

The predefined volume of aerosolized particles is delivered into the flow path through which the patient is inhaling. Inhalation time is preset to comprise a three predefined periods.

The first predefined time period is for delivery of aerosol particle free air into the lungs at a flow rate that is also preset.

The second predefined period is for delivery of a predefined volume of aerosolized particles of the steroid/methylxanthine combination, also at a preset flow rate.

The third predefined period is for delivery of the second predefined time period of a particle free air.

Optionally, the first time period can be set to zero seconds, meaning that the aerosolization will start immediately without the delivery of the particle free air.

During the inhalation, patient is instructed to begin inhalation and during each inspiration time, the three (or two) predetermined periods are repeated. At the end of the second particle free period, that is, after the second predefined period, a patient is instructed to stop inhaling and exhale. The reason for the second predefined time period of aerosol particle free air delivery into the lungs at a flow rate within the preset flow rate range is to move the aerosolized particles out of the upper airway region. In that way the upper airway region (mouth, throat, oropharynx and larynx) is emptied from remaining aerosol particles and the deposition of the drug in this region is reduced. This will reduce oropharyngeal deposition and bitter taste, cough and bronchospasm.

The method additionally comprises a step of detecting when the subject is inhaling through the flow path and may further comprise steps of measuring and adapting the first, the second and the third predefined time period and/or the predefined volume of aerosolized particles to patient's health parameters.

The method determines optimal time intervals for administration of the first particle-free air, for administration of an aerosolized inhalable steroid/methylxanthine combination and for administration of the second particle free air, wherein the cumulative time for these three time intervals corresponds to one inspiration time. The time for each of the interval corresponds to from about 1 msec to about 10 sec, preferably from about 200 msec to about 5 seconds, and may be the same or different for each interval.

The flow rate is a predetermined fixed flow rate, wherein the first predefined particle free air volume is up to about 0.15 liters, the predefined volume of aerosolized particles is up to about 3 liters and the second predefined particle free air volume is up to about 0.5 liters.

The nebulizer used for this method is equipped to detect when the subject is inhaling through the flow path and prevent flow through the flow path after providing the second predefined time period of aerosol particle free air.

IV. Devices and Properties Thereof

Devices suitable for practicing the current invention have to have certain properties that meet the criteria for delivery of inhalable steroid/methylxanthine combination to the central and conducting airways according to the invention.

Aerosolization of methylxanthines, such as theophylline and aminophylline, is problematic and typically results in cough and bronchospasm (Thorax, 40: 176-179 1985)) when delivered with conventional nebulizers. Only novel approaches that utilize vibrating mesh nebulizers, produce monodisperse particle sizes, along with specific airflow control and control breathing pattern of the patient, are able to deposit sufficient methylxanthine amounts into the lung.

Monodisperse particle size with a GSD, geometric standard deviation, of 1.6 to 2 μm can be achieved by using the vibrating mesh nebulizers combined with the airflow control achieved by the AKITA®1 and 2. In combination, the monodisperse particle spectrum, along with controlled airflow overcomes the problematic oropharyngeal side effects of the inhaled methylxanthine.

Additionally, the devices suitable for practicing the current invention are the new, handheld breath and airflow control devices embracing AKITA® nebulizer principles. These devices are typically miniaturized to be able to be handheld.

These devices are, for example, Fox-POP®™, Medspray™ and Telemag™ handheld nebulizers commercially available or soon to be available from Activaero GmbH, Gemünden (Wohra), Germany or are currently in development. Fox-POP handheld mininebulizer disclosed in U.S. application Ser. No. 12/183747, filed on Jul. 31, 2008, publication number 2009/0056708, herein incorporated by reference in its entirety. Another suitable minidevice is Medspray disclosed in WO 2006/094796, hereby incorporated by reference in its entirety.

Device for Control of Breathing Pattern

The device for control of breathing pattern is suitable for practicing the current invention is an inhalation system that comprises a compressor-driven jet nebulizer that controls the patient's breathing pattern during the inspiration phase. This system is highly effective for inhalation therapy requiring deposition of the aerosol into the lower lungs. During the inhalation, the system controls the number of breaths, the flow rate and inspirational volume. This ability to control these three parameters assures that the patient is given a correct dose.

The system further comprises an electronic means for individual personalization of a treatment protocol. The treatment protocol includes such parameters as individual's lung function measurements, optimum breathing pattern, desired drug dose to maintain or restore patients vital capacity (VC), expiratory resting volume (ERV) and forced expiratory volume per one second (FEV1). These parameters are individualized and stored on individual electronic record, called Smart Card. The electronic records not only store the information for a treatment protocol and transfer this information to the system during treatment but also record and store the information for each of the treatments and show a possible error.

The Smart Card system may hold more than one treatment configuration and is fully encrypted. The Smart Card system is disclosed in the co-pending US patent application 2001/0037806 A1, published on Nov. 8, 2001, herein incorporated by reference in its entirety. The same or similar nebulizing system is disclosed in the U.S. Pat. No. 6,606,989, herein incorporated by reference in its entirety and is commercially available from Activaero GmbH, Gemünden (Wohra), Germany, under the trade name AKITA® Inhalation System.

A similar but modified device for the inhalation system further comprises, as a core element, a circular perforated membrane that may be set to vibrate by a piezo-electric actuator. The vibrating motion of the membrane generates an alternating pressure that forces the nebulizing solution through a microarray of perforation in the membrane thus creating a fine aerosol having defined particle sizes. This system is similarly equipped with electronic means comprising the Smart Card, as described above. This system is commercially available from Activaero GmbH, Gemünden (Wohra), Germany, under the trade name AKITA² APIXNEB Inhalation System.

Another device comprising modifications of the inhalation system that can be used for practicing the current invention is the nebulizer that is triggered by the negative trigger pressure detected by a pressure sensor. This nebulizer comprises a compressor that provides a constant inhalation flow rate of 12 liters/minute during inspiration and has a controlled flow, volume and nebulization timing. The Smart Card settings include inhalation volume, inhalation time per breath, nebulization time per one breath. This system is commercially available from Activaero GmbH, Gemünden (Wohra), Germany, under the trade name AKITA JET Inhalation System.

Other inhalation devices and systems that may be conveniently used or modified for use by the current invention are disclosed in the U.S. Pat. Nos. 6,401,710 B1, 6,463,929 B1, 6,571,791 B2, 6,681,762 B1 and 7,077,125 B2 or in published applications 2006/0201499 A1 and 2007/0006883 A1, all herein incorporated by reference in their entirety.

B. Breath Actuated Nebulizer Device

Another type of device suitable for practicing the current invention is a breath actuated nebulizer. This nebulizer is characterized by a passive flow and active volume control. Typically, it comprises a single use aerosol generator and a multi-use control device.

The device consists of an inhaler that is connected with a control unit. Inhaler itself is connected with nebulizer where the inhalable methylxanthine, such as theophylline and aminophylline, in combination with a steroid is nebulized into predetermined particles having sizes predominantly in the range from about 3 to about 8 μm, MMAD, using an aerosol generator. The filling volume of the nebulizer is approximately 2-4 ml. The aerosol generator is activated by pressure detection and is only activated during inspiration phase when the patient is inhaling the aerosolized methylxanthine/steroid combination. The pressure detection is controlled electronically.

This device is further equipped with means to permit administration of particle-free air, to permit the administration of an aerosolized inhalable methylxanthine/steroid combination, and to permit the second administration of the particle free air, each for a preselected time and volume, wherein the cumulative time for these three time intervals correspond to one inspiration time. The time for each of the interval corresponds to from about 1 msec to about 10 sec, preferably from about 200 msec to about 5 seconds.

The inhaler has an integrated flow and volume limited to about 15 liters/minute flow at a pressure of about 10 mbar or lower. When the underpressure at the mouthpiece is below 5 mbar, the flow rate is limited by a mechanical valve. The mechanical valve regulates the flow rate by a adjusting the cross section area. The unit is preset to a volume per one breath. One breath is set to be a time when one inspiration and one expiration occur. After each inspiration time, the inspiration flow is blocked and expiration allowed. The inspiration flow is restored again for the next inspiration time during the next breath.

This device has various electronic components that permit its preprogramming and individualization meeting requirements of the individual asthmatic patients.

The modified device and method for its use is disclosed in the U.S. application Ser. No.: 12/204,037, herein incorporated by reference in its entirety.

V. Advantages of the Treatment of Pulmonary Diseases

The method for treatment of pulmonary diseases, such as chronic pulmonary disease, asthma, cystic fibrosis and idiopathic pulmonary disease according to the current invention provides several advantages over the currently available treatments.

The method for treatment of pulmonary diseases according to the current invention provides a substantial improvement in efficacy of the combination drug delivery compared to the currently available conventional treatments, first by delivering the combination of two drugs in one aerosolization, second by delivering four to five time more of this combination into the patient lungs in shorter nebulization time and third by eliminating secondary side effects previously observed with delivery of these two drugs.

The method for treatment of pulmonary diseases according to the current invention allows a deposition of high doses of methylxanthine/steroid combination in the central and conducting airways of the lungs of patients, with a concurrent reduction in oropharyngeal side effects, due to a targeted and selective deposition of the aerosolized particles of the drug combination into the targeted airways due to the slow and regulated breathing pattern.

The method further provides an aerosol having the optimal particle sizes for homogenous deposition of the drug in the central and conducting airways of the lungs that prevents high losses of drug in the oropharynx, larynx and mouth. The method further reduces the deposition variability previously seen with conventional nebulizers (such as, e.g. Pari Jet nebulizers).

The method for treatment of pulmonary diseases according to the current invention also provides for administration of the aerosol, during inhalation, under a mild or moderate controlled overpressure to allow preferable deposition of the aerosolized drug into the central and conducting airways of the lungs and prevent exhalation of aerosol during the exhalation phase.

The combination of two drugs or the prodrug masks the pharmacologic properties of methylxanthines and steroids, thereby eliminating or greatly reducing cough, bronchospasm, dysphonia and other side effects in the oral pharyngeal cavity. The combination also masks the methylxanthine activity minimizing a chance for systemic cardiovascular and central nervous side-effects.

### Utility

The compounds of the invention are useful for treating pulmonary inflammation and bronchoconstriction. The aim of these treatments is to overcome steroid resistance that develops in pulmonary diseases. Inhaled steroids are not as effective in treatments of all pulmonary disease as they are in treatment of asthma and, consequently, the reversing effect of theophylline and other methylxanthines for such steroid resistance needs to be affected directly in the lungs.

The combination of the steroid and methylxanthine, particularly theophylline, both in much smaller concentrations than currently used provides means or overcoming such resistance to steroid treatment observed in pulmonary diseases. This small volume, high concentration aerosolable formulation of steroid/methylxanthine or its prodrug is delivered as an aerosol and at efficacious concentrations to the respiratory tract in patients suffering from mild to severe COPD, asthma while smoking, chronic bronchitis, cystic fibrosis and idiopathic pulmonary fibrosis. The combination of the steroid with methylxanthine may be advantageously formulated as a solid dosage formulation that is stable, readily manufactured with adequate shelf life for commercial distribution and that is very cost effective.

### EXAMPLE 1

### Theophylline/Fluticasone Combination Solution for Inhalation Used for Treatment of COPD Patients

This example describes a clinical trial with inhalable theophylline (7.5 mg/mL, 2 mL, plus fluticasone 500 ug, BID), versus fluticasone 500 ug alone in 2 mL, BID, versus placebo BID, for treatment of subjects with COPD. The clinical trial is performed in a double blinded, three arm, placebo controlled study in patients with COPD.

For the trial, inhalable theophylline (7.5 mg/mL, plus fluticasone 500 ug, in 2 mL), versus fluticasone 500 ug alone in 2 mL, versus placebo (2 mL of isotonic saline) are delivered via AKITA-FOX electronic nebulizer with airflow control. All inhalation treatments are administered twice daily (BID).

COPD per GOLD inclusion criteria (the same number of females and males, 18 to 65 years of age, having FEV1 40-80%) are enrolled, randomized to three groups, and treated with two doses daily for four weeks. The full individual dose of 2 ml is administered in 3-4 minutes treatment time.

Airway irritation and acute bronchospasm are assessed by measuring spirometry immediately prior to and 30 min post-completion of aerosol administration at the first dose. A decrease in forced expired volume in one second (FEV1) >20% in the 30 minutes spirometry test are considered evidence of bronchospasm. All patients are tested 14 and 28 days into the study for FEV1, FVC, and 6 minute walk distance, and Quality of Life Questionnaire (St. George's Questionnaire).

Safety endpoints are FEV1, systemic (blood) and urine levels of theophylline, taste, GI symptoms, other adverse Events.

Efficacy endpoints are pulmonary function (FEV1), measured upon first dose at two hours, and at 14 and 28 days; and exhaled NO (FeNO) change expressed as a percentage of increase, compared to baseline. Mean changes in both 6 minute walk test as well as FEV1 are compared between theophylline/fluticasone combination compared to placebo (primary efficacy analysis) as well as fluticasone alone compared to placebo.

### EXAMPLE 2

### Nebulization of Methylxanthine/Steroid Combination

A methylxanthine/steroid combination is prepared according to Example 1. The AKITA® nebulizer (AKITA-FOX device) is connected to an airflow control or triggered release device. Other nebulizer may be used instead if it meets requirements of this invention. Because of its consistent, less variable deposition variability, the AKITA-FOX device is preferred.

The methylxanthine/steroid combination or the steroid/methylxanthine prodrug combination is nebulized using said nebulizer and nebulizing protocol permitting a slow breathing pattern and administration of bolus of said combination. The amount of methylxanthine and steroid released from the formulation into the lungs and into plasma is determined.

What is claimed:

1. A method for treatment of pulmonary diseases comprising steps:
preparing a suspension comprising a drug combination of a methylxanthine and a topical steroid, a methylxanthine prodrug and a steroid, or a methylxanthine alone, wherein said suspension comprises from about 0.1 mg to about 2 mg of said steroid and from about 25 to about 50 mg of said methylxanthine dissolved in from about 1 to about 3 mL of a solvent;
aerosolizing said suspension into an aerosol having particle sizes between about 3 and about 8 μm MMAD;
administering said aerosol to a patient in need thereof using a nebulizing system comprising an electronic or jet nebulizer, a compressor and an electronic control means for controlling an airflow, breathing pattern of a patient and delivery of the aerosol as a bolus according to a treatment protocol, said treatment protocol providing for a slow and controlled breathing pattern of the patient, for controlled airflow of the air or aerosol, for bolus drug delivery and for delivery of said aerosol with efficacy of about 60% to about 70% predominantly into a conducting and central airway with overpressure of 30 mbar or less under controlled conditions; and
delivering said drug combination according to said protocol predominantly into patient conducting and central airways with efficacy of at least 60% deposition of said aerosol in the conducting and central airways,
wherein said treatment results in improvement of pulmonary functions measured by FEV1 and in reduction of oropharyngeal deposition and reduction in methylxanthine or steroid side effects.

2. The method of claim 1 wherein said pulmonary disease is chronic obstructive pulmonary disease, asthma, steroid dependent asthma, asthma in smokers or in subjects subjected to secondary smoke, cystic fibrosis, idiopathic pulmonary fibrosis or pulmonary arterial hypertension.

3. The method of claim 2 wherein the methylxanthine is selected from the group consisting of theophylline, aminophylline, enprophylline, pentoxyphylline, diprophylline and a phosphodiesterase inhibitor.

4. The method of claim 3 wherein said steroid is selected from a group consisting of prednisone, fluticasone, beclomethasone, budenoside, mometasone and ciclesonide.

5. The method of claim 4 wherein said combination comprises from about 0.1 mg to about 2 mg of said steroid and from about 25 to about 50 mg of said methylxanthine, in combination, dissolved in from about 1 to about 3 ml of solvent of which at least 1 mL comprising at least 0.1 mg of steroid and at 2 to 15 mg of methylxanthine per treatment is deposited in the conducting and central lungs.

6. The method of claim 1 wherein said nebulizer is the jet nebulizing device.

7. The method of claim 1 wherein said nebulizer is electronic nebulizer.

8. The method of claim 7 wherein said electronic nebulizer further comprises a vibrating mesh or vibrating membrane.

9. The method of claim 1 wherein said treatment is accomplished in less than 15 minutes.

10. The method of claim 1 wherein at least 90% of said particle have sizes between 3 and 8 μm MMAD with GDS of between 1.6 and 2.25.

11. The method of claim 1 wherein said aerosol is administered predominantly into the conducting and central airways with overpressure of about 10 to about 20 mbar under controlled conditions comprising a slow inhalation breathing pattern combined with an aerosol bolus delivery and wherein such delivery results in deposition of at least 1 ml of the aerosolized suspension.

12. The method of claim 11 wherein the methylxanthine is theophylline, wherein a nominal dose of theophylline is between about 3 and about 50 mg and wherein at least 0.1 mg of the steroid and at least 2 mg of theophylline is deposited in the conducting and central airways.

13. The method of claim 12 wherein said treatment is administered once or twice a day.

14. The method of claim 13 wherein said treatment is accomplished in between four and ten minutes.

15. The method of claim 14 wherein said aerosol is administered during an inspiration time comprising three predefined periods,
wherein in the first period lasting from about 1 millisecond to about 1 second, an aerosolized particle free air is administered at a preset flow rate and at a preset volume;
wherein in the second period lasting from about 0.1 to about 7 seconds, the aerosolized drug combination is administered at a preset flow rate and at a preset volume;
wherein in the third period, lasting from about 1 millisecond to about 10 seconds, an aerosolized particle free air is administered at a preset flow rate and at a preset volume;
wherein after the third period, the patient is instructed to stop inhaling and exhale;
wherein said protocol is repeated for from about 4 to about 15 minutes.

16. The method of claim 15 wherein said preset flow rate is an inspirational flow rate and is equal or below 20 liters min.

17. The method of claim 16 wherein said aerosolized particle free air administered in the first period is administered at a preset volume of less than 150 ml in about 0.5 second time.

18. The method of claim 17 wherein said aerosol administered in the second period is administered at a volume of from about 200 to about 2000 ml or in a preset time of from 1 to about 7 seconds.

19. The method of claim 18 wherein said aerosolized particle free air administered in the third period is administered at a preset volume from about 200 to about 500 ml in about 0.3 to about 3 seconds time.

20. The method of claim 19 wherein said aerosol administered during the inspiration time and comprising three predefined periods is generated by a breath actuated nebulizer.

21. The method of claim 1 wherein said nebulizer is a handheld nebulizer.

22. The method of claim 1 wherein said drug combination comprises the methylxanthine prodrug.
